(19) United States
(12) Patent Application Publication
(10) Pub. No.: US 2014/0221506 A1
(43) Pub. Date: Aug. 7, 2014

(54) Methods of Diagnosing Neuropathic Pain

(71) Applicants: Drexel University, Philadelphia, PA (US); PHILADELPHIA HEALTH & EDUCATION CORPORATION, Philadelphia, PA (US)

(72) Inventors: Seena Ajit, West Windsor, NJ (US); Ahmet Sacan, Drexel Hill, PA (US); Guillermo Alexander, Bryn Mawr, PA (US); Robert J. Schwartzman, Bryn Mawr, PA (US)

(73) Assignee: PHILADELPHIA HEALTH & EDUCATION CORPORATION, Philadelphia, PA (US)

(21) Appl. No.: 14/117,735

(22) PCT Filed: May 18, 2012
(86) PCT No.: PCT/US12/38550

§ 371 Date: Apr. 21, 2014

Related U.S. Application Data
(60) Provisional application No. 61/487,560, filed on May 18, 2011.

(51) Int. Cl. C12Q 1/68

(52) U.S. Cl. CPC C12Q 1/6883
USPC 514/789; 435/6.12; 506/9; 435/6.11

(57) ABSTRACT

The invention includes compositions and methods useful for the diagnosis, assessment, and characterization of neuropathic pain in a subject in need thereof, based upon the expression level of at least one miRNA or snoRNA that is associated with neuropathic pain.

### BACKGROUND OF THE INVENTION

Neuropathic pain arises from nerve damage or dysfunction, adversely impacting quality of life and imposing a large healthcare burden (O'Connor et al., 2009, Am. J. Med. 122:S22-32; Seal et al., 2009, Nature 462:651-655; Costigan et al., 2009, Annu. Rev. Neurosci. 32:1-32). A deeper understanding of the molecular mechanisms underlying neuropathic pain could provide a first step toward the development of better treatment options for patients. A frequently used rat model to study the molecular mechanisms of neuropathic pain is spinal nerve ligation (SNL) wherein one or more spinal nerves innervating the hind limb are ligated (Kim et al., 1992, Pain 50:355-363), typically unilaterally. The injury, which results in hyperalgesia, an enhanced response to mechanical stimuli, has a well-characterized time course. Since they represent a primary site for pain processing, dorsal root ganglion (DRG) neurons have been the focus of much research to identify molecular targets of pain neurotransmission. Previous studies using animal pain models have measured mRNA levels by examining a targeted set of transcripts or through the use of global approaches such as microarray technology to study mRNA expression changes (Xiao et al., 2002, Proc. Natl. Acad. Sci. USA 99:8360-8365; Costigan et al., 2002, BMC Neurosci. 3:16). In a proteomic study, 67 proteins have been shown to be regulated in the SNL model (Komori et al., 2007, Physiol. Genomics 29:215-230).

Complex regional pain syndrome (CRPS) is a disabling chronic neuropathic pain syndrome that can affect one or more extremities. The broad spectrum of symptoms includes pain, inflammation, sensory dysfunction, impaired motor function, and trophic disturbances (Schwartzman, et al., 2006, Expert Rev. Neurother. 6:669-681; Schwartzman et al., 2009, Clin. J. Pain 25:273-280; Costigan et al., 2009, Annu. Rev. Neurosci. 32:1-32; Maihofner et al., 2010, Eur. J. Neurol. 17:649-660). CRPS is subdivided into CRPS-I (reflex sympathetic dystrophy) and CRPS-II (causalgia), based on the absence or presence of documented nerve injury respectively (Harden et al., 2007, Pain Med. 8:326-331). The complex multifactor pathogenesis of CRPS includes inflammatory, vascular, sympathetic nervous system, cortical, and spinal mechanisms. The pathophysiology of CRPS is not completely understood and the diagnosis is based solely on clinical observations. Not all disease mechanisms are equally prominent in each patient and no single therapeutic modality is sufficient to attenuate all of the symptoms.

MicroRNA (miRNA), a class of 22 nucleotide, non-protein encoding endogenous RNA molecules, has attracted considerable attention recently for its role in the molecular changes underlying various disease models (Erson et al., 2008, Clin. Genet. 74-296-306). miRNAs participate in the regulation of gene expression by binding to the 3′ untranslated region (3′-UTR) of target mRNAs, which can result in reduced expression of the proteins encoded by such target RNAs. Reduction of protein expression can come about by either of two mechanisms, the cleavage and degradation of the mRNA target or repression of its translation. Under the former mechanism but not the latter, an inverse correlation between miRNA and target mRNA expression is expected. Each miRNA species regulates multiple genes, and most mRNAs contain multiple miRNA binding sites within their 39-UTR, suggestive of a complex regulatory network (Bartel, 2009, Cell 136:215-233). Aberrant miRNA expression is a feature in a variety of human diseases. Understanding the gene regulation events in neuropathic pain mediated by miRNAs could provide an avenue for the identification of biomarkers or discovery of novel therapeutic targets (Erson et al., 2008, Clin. Genet. 74:296-306).

miRNAs play important roles in the regulation of gene expression and function by binding to the 3+ untranslated region (3′-UTR) of target messenger RNAs (miRNAs) which causes cleavage or repression of translation of these mRNAs. Each miRNA species regulates multiple genes, and most mRNA targets contain multiple miRNA binding sites within their 3′-UTR, suggesting a complex regulatory network (Bartel, 2009, Cell 136:215-233). As aberrant miRNA expression is a common feature in a variety of human diseases, these molecules offer novel avenues for the identification of biomarkers and new opportunities for the discovery and validation of novel therapeutic targets (Ceman et al., 2011, Pharmacol. Ther. 130:26-37).

It was recently demonstrated that miRNAs are present in the serum and plasma of humans and other mammals, such as rats, mice, cows and horses (Chen et al., Cell Res. 18:997-1006; Mitchell et al., 2008, Proc. Natl. Acad. Sci. USA 105:10513-10518). This finding opens up the feasibility of using miRNAs as biomarkers of disease. Though the stability of miRNAs in scrum was the initial concern, it has now been demonstrated that these circulating miRNAs are protected from plasma RNase activity and are, in fact quite stable (Chen et al., 2008, Cell Res. 18:997-1006). The existence of tumor-related miRNAs in serum indicates the potential usefulness of miRNAs as clinical diagnostic biomarkers of various cancers (Cortez et al., 2009, Expert Opin. Biol. Ther. 9:703-711). In another recent report, dozens of stable miRNAs were detected in saliva and two miRNAs were present in significantly lower levels in the saliva of patients with oral squamous cell carcinoma compared to control subjects (Park et al., 2009, Clin. Cancer Res. 15:5473-5477). Further evidence for the presence of miRNAs in body fluids came from an analysis of urine samples (Gilad et al., 2008, PLoS One 3:e3148). Four miRNAs were significantly elevated in urine from urothelial bladder cancer patients, demonstrating the utility of miRNAs as a noninvasive diagnostic option (Hanke et al., 2009, Urol. Oncol.). All of these studies illustrate the potential use of miRNAs as novel biomarkers amendable to clinical diagnosis in translation medicine (Gilad et al., 2008, PLoS One 3:e3148; Weber et al., 2010, Clin. Chem. 56:1733-1741; Etheridge et al., 2011, Mutat. Res.; Scholer et al., 2010, Exp. Hematol. 38:1126-1130). Biomarkers can be used to determine the propensity to develop a disease, measure its progress, or predict prognosis (Wehling, 2006, Eur. J. Clin. Pharmacol. 62:91-95). In clinical trials, biomarkers can help in patient stratification and thereby increase the chances of a successful outcome by targeting the appropriate population, In addition, biomarkers can pave the way to individualize treatment and thereby usher in a new era in personalized medicine (Frank et al., 2003, Nat. Rev. Drug Discov. 2:566-580).

A number of studies have addressed miRNA changes in rodent models of inflammatory and neuropathic pain indicating an essential role for miRNAs in altering pain threshold (Bai et al., 2007, Mol. Pain 3:15; Kosuka et al., 2001, Mol. Pain 7:17; Poh et al., 2011, Eur. J. Pain 15:801, e801-812; Aldrich et al., 2009, Neuroscience 164:711-723; von Schack et al., 2011, PLoS One 6:e17670; Zhao et al., 2010, J. Neurosci. 30:10860-10871; Favereaux et al., 2011, EMBO J. 30:3830-3841). Although deregulation of miRNA is now a well-established phenomenon in a number of human diseases including osteoarthritis pain (Li et al., 2011, Gene 480:34-41), the role of miRNA expression inpatients with neuropathic pain is unknown.

It has been shown that miRNAs are transported in plasma and delivered to recipient cells by high-density lipoproteins resulting in modulation of target mRNAs (Vickers et al., 2011, Nat. Cell Biol. 13:423-433). One mode of miRNA transport in body fluids is via exosomes. Exosomes are small vesicles originating from the inward budding of the plasma membrane. They carry mRNAs, miRNAs, proteins and lipid mediators to recipient cells with functional gene regulatory consequences via blood indicating a novel mechanism of cellular communication. Depending on their origin, exosomes have been shown modulate immune-regulatory processes and are known to contain interleukin-1beta, TNFalpha or TGFbeta (Thery et al., 2009, Nat. Rev. Immunol. 9:581-593). Target cell-specific receptors guide exosomes, and information transfer occurs upon fusion with the plasma membrane of the recipient cells.

there is thus a need in the art for compositions and methods for detecting and quantifying miRNA expression for the assessment and characterization of neuropathic pain in a subject. The present invention addresses this unmet need in the art.

### SUMMARY OF THE INVENTION

The present invention relates to the discovery that the expression levels of some microRNAs (miRNAs) and small nucleolar RNAs (snoRNAs) are associated with neuropathic pain. Thus, in various embodiments described herein, the methods of the invention relate to methods of diagnosing a subject as having neuropathic pain, methods of assessing a subject's risk of having or developing neuropathic pain, methods of assessing the severity of a subject's neuropathic pain, and methods of stratifying a subject having neuropathic pain for assignment in a clinical trial.

In one embodiment, the invention is a method of diagnosing neuropathic pain in a subject including the steps of obtaining a biological sample from the subject, determining the level of at least one microRNA or snoRNA in the biological sample, comparing the level of the at least one microRNA or snoRNA in the biological sample with the level of the at least one miRNA or snoRNA in a comparator, wherein when the level of the at least one microRNA or snoRNA in the biological sample is different than the level of the at least one miRNA or snoRNA in the comparator, the subject is diagnosed with neuropathic pain. In some embodiments, the neuropathic pain is complex regional pain syndrome (CPRS). In various embodiments, the at least one microRNA or snoRNA is at least of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In preferred embodiments, the subject is human. In some embodiments, the method includes the additional step of treating the subject for neuropathic pain. In various embodiments, the comparator is at least of a positive control, a negative control, a normal control, a wild-type control, a historical control, and a historical norm. In various embodiments, the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%. In other various embodiments, the level of the at least one miRNA or snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, or by at least 100%. In some embodiments, the step of determining the level of the at least one microRNA or snoRNA utilizes at least one technique selected from the group consisting of reverse transcription, PCR and a microarray.

In another embodiment, the invention is a method of determining the severity of neuropathic pain in a subject including the steps of obtaining a biological sample from the subject, determining the level of at least one microRNA or snoRNA in the biological sample, comparing the level of at least one microRNA or snoRNA in the biological sample, comparing the level of the at least one microRNA or snoRNA in the biological sample with the level of the at least one miRNA or snoRNA in a comparator, wherein the greater the difference between the level of the at least one microRNA or snoRNA in the biological sample and the level of the at least one miRNA or snoRNA in the comparator, the greater the severity of neuropathic pain. In some embodiments, the neuropathic pain is complex regional pain syndrome (CPRS). In various embodiments, the at least one microRNA or snoRNA is at least of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-531-3p, hsa-miR-181a-2#, RNU48, hsa-miR-720, RNU44, and hsa-miR-1201. In preferred embodiments, the subject is human. In some embodiments, the method includes the additional step of treating the subject for neuropathic pain. In other embodiments, the method includes the additional step of stratifying the subject for inclusion in a clinical trial based upon the severity of the subject's neuropathic pain. In various embodiments, the comparator is at least of a positive control, a negative control, a normal control, a wild-type control, a historical control, and a historical norm. In various embodiments, the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%. In other various embodiments, the level of the at least one miRNA or snoRNA is lower than the level of the at least one miRNA or snoRNA in the comparator by at least 10%, by at least 20%, by at least 30%, by at least 40%, by at least 50%, by at least 60% by at least 70%, by at least 80%, by at least 90%, or by at least 100%. In some embodiments, the step of determining the level of the at least one microRNA or snoRNA utilizes at least one technique selected from the group consisting of reverse transcription, PCR and microarray.

In a further embodiment, the invention is a method of evaluating the progression of neuropathic pain in a subject including the steps of obtaining a biological sample from the subject, determining the level of at least one microRNA or snoRNA in the biological sample at a first time point, comparing the level of the at least one microRNA or snoRNA in the biological sample at the first time point with the level of the at least one miRNA or snoRNA in a comparator, determining the level of at least one microRNA or snoRNA in the biological sample at a second time point, comparing the level of the at least one microRNA or snoRNA in the biological sample at the second time point with the level of the at least one miRNA or snoRNA in a comparator, wherein when the difference in the level of the at least one microRNA or snoRNA in the biological sample at the second time point, as compared with the comparator, is greater than the difference in the level of the at least one microRNA or snoRNA in the biological sample at eh first time point, as compared with the comparator, the neuropathic pain is progressing. In some embodiments, the neuropathic pain is complex regional pain syndrome (CPRS). In various embodiments, the at least one microRNA or snoRNA is at least of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In preferred embodiments, the subject is human. In some embodiments, the method includes the additional step of treating the subject for neuropathic pain. In other embodiments, the method includes the additional step of modifying the subject's treatment for neuropathic pain. In various embodiments, the comparator is at least of a positive control, a negative control, a normal control, a wild-type control, a historical control, and a historical norm. In various embodiments, the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 5000%. In other various embodiments, the level of the at least one miRNA snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%. In some embodiments, the step of determining the level of the at least one microRNA or snoRNA utilizes at least one technique selected from the group consisting of reverse transcription, PCR and a microarray.

In yet a further embodiment, the invention is a method of evaluating a treatment of neuropathic pain in a subject in need thereof including the steps of obtaining a biological sample from the subject, determining the level of at least one microRNA or snoRNA in the biological sample at a first time point, comparing the level of the at least one microRNA or snoRNA in the biological sample at the first time point with the level of the at least one miRNA or snoRNA in a comparator, determining the level of at least one microRNA or snoRNA in the biological sample at a second time point, comparing the level of the at least one microRNA or snoRNA in the biological sample at the second time point with the level of the at least one miRNA or snoRNA in a comparator, wherein when the difference in the level of the at least one microRNA or snoRNA in the biological sample at the second time point, as compared with the comparator, the treatment of neuropathic pain is reducing neuropathic pain, In some embodiments, the neuropathic pain is complex regional pain syndrome (CPRS). In various embodiments, the at least one microRNA or snoRNA is at least of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In preferred embodiments, the subject is human. In some embodiments, the method includes the additional step of continuing to treat the subject for neuropathic pain. In other embodiments, the method includes the additional step of modifying the subject's treatment for neuropathic pain. In various embodiments, the comparator is at least of a positive control, a negative control, a normal control, a wild-type control, a historical control, and a historical norm. In various embodiments, the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%.by at least 700%, by at least 800%, by at least 900%, by at least 1000%, by at least 1500%, by at least 2000%, or by at least 5000%. In other various embodiments, the level of the at least one miRNA or snoRNA is lower than the level of the at least one miRNA or snoRNA in the comparator by at least 10%, by at least 20%, by at least 30%, by at least 40%, by at least: 50%, by at least 60%, by at least 70%, by at least 80%, by at least 90%, or by at least 100%. In some embodiments, the step of determining the level of the at least one microRNA or snoRNA utilizes at least one technique selected from the group consisting of reverse transcription, PCR and a microarray.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the results of experiments assessing the differential expression of miRNAs in blood from CRPS patients and control samples. A clustergram of the samples and miRNAs identifying sample subpopulations with respect to the significant differentially expressed miRNAs are depicted. A total of 60% of the patients (Group 3) showed differential regulation for these miRNAs (mostly downregulation), whereas the rest of the patients were more heterogeneous and clustered together with the control samples. P and C represent patients and controls, respectively.

FIG. 2, comprising FIGS. 2A-2C, is a series of graphs depicting the elevation of inflammatory markers in CRPS patients. VEGF (FIG. 2A), IL1Ra (FIG. 2B), and MCP1 (FIG. 2C) showed significant differential expression in CRPS patients vs. control samples, with p values 0.0002, 0.0002, and 0.0005, respectively. The error bars show the standard error of the means.

FIG. 3 is a Circos diagram showing the correlation of selected parameters and miRNAs. The Circos diagram showing the correlation of selected medical conditions with other clinical parameters and miRNAs. The nodes along the circle are colored by the total strength of correlation of the corresponding variable. The links between variables indicate their correlation. Only correlations with adjusted p value less than 0.01 are shown. Groups of miRNAs showing similar correlations with other variables are labeled as “miRNAs” for brevity. The links are colored by the Spearman's correlation value, with strong negative correlations shown as implicated by the color-bar (e.g., narcotics vs. hsa-miR-191) and strong positive correlations shown as implicated by the color-bar (e.g., pain level vs. “IL1ra, VEGF, miRNAs”). See FIG. 7 for a complete list of correlations.

FIG. 4 is a table depicting fold changes and p values of significantly altered miRNAs

FIG. 5 is a table listing significantly altered miRNAs.

FIG. 6 is a table depicting correlation studies in CRPS patients of miRNAs, comorbidities and medication.

FIG. 7 is a table depicting correlation studies of miRNAs, comorbidities and medication. Significant correlations observed including 1) miRNA and clinical parameters, 2) miRNA and inflammatory markers, 3) miRNA and groups, 4) inflammatory and immune related markers, and 5) clinical parameters are shown in sheets 1, 2, 3, 4 and 5 respectively. Groups in sheet 3 includes CRPS vs. healthy as well as CRPS subpopulation 1, CRPS subpopulation 2 and CRPS subpopulation 3 representing group 1, 2 and 3 based on differential expression of miRNAs. miRNAs differentially expressed in CRPS are highlighted (shown in FIG. 4).

FIG. 8 depicts the results of an experiment assessing the presence of HSP70 in exosomes isolated from HEK293 cells.

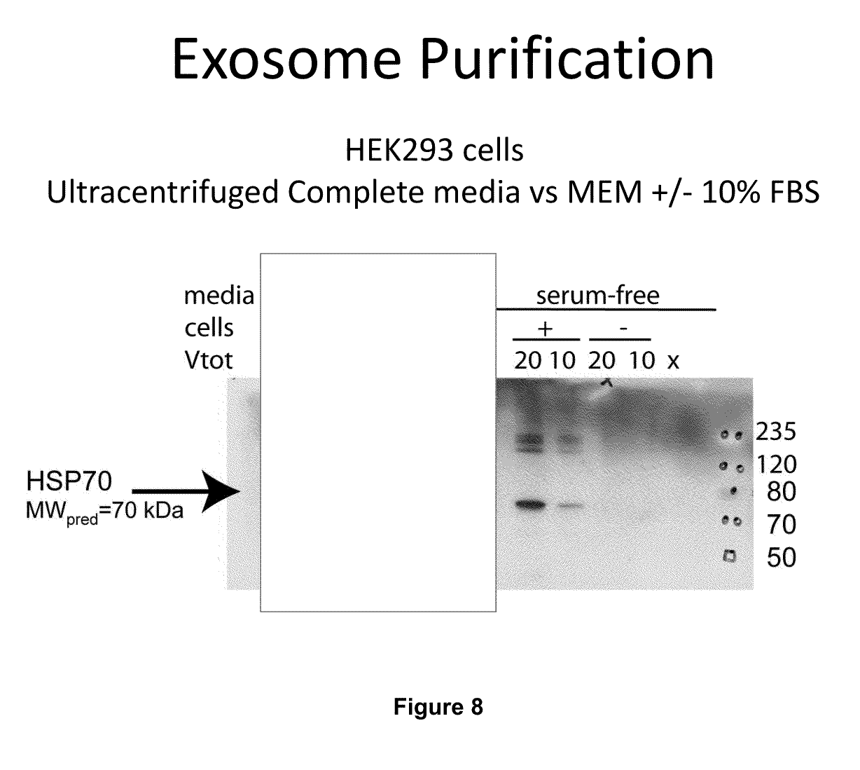

FIG. 9 depicts the results of an experiment assessing the presence of HSP70 is exosomes isolated from RAW 264.7 cells and human aortic endothelial cells.

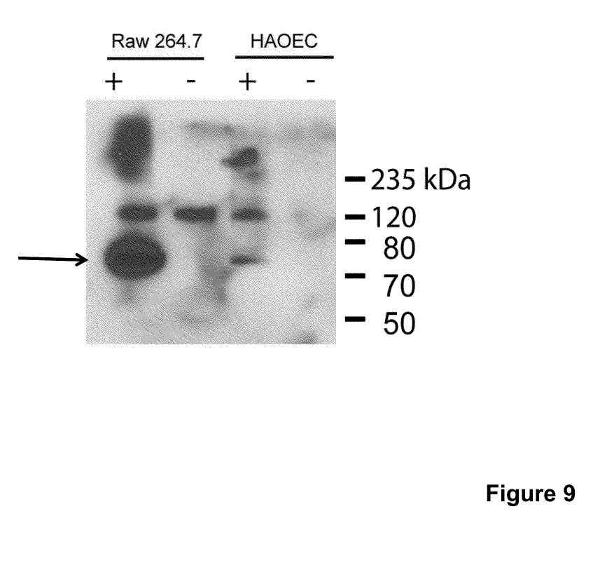

FIG. 10 depicts the results of an experiment assessing exosomes purified from mouse blood.

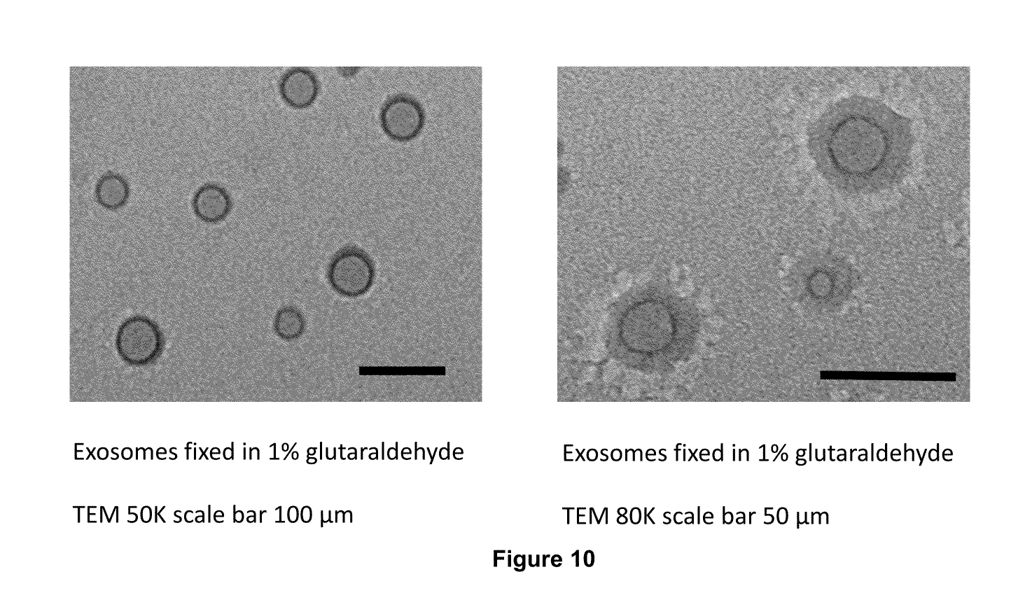

FIG. 11 depicts the results of an experiment assessing miRNAs in purified exosomes.

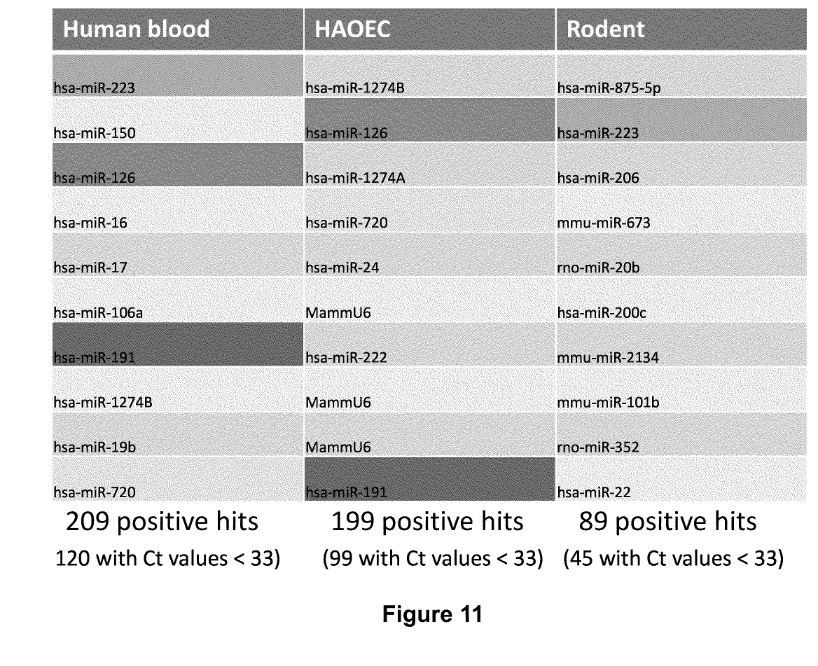

FIG. 12 is a table listing putative targets of mir939.

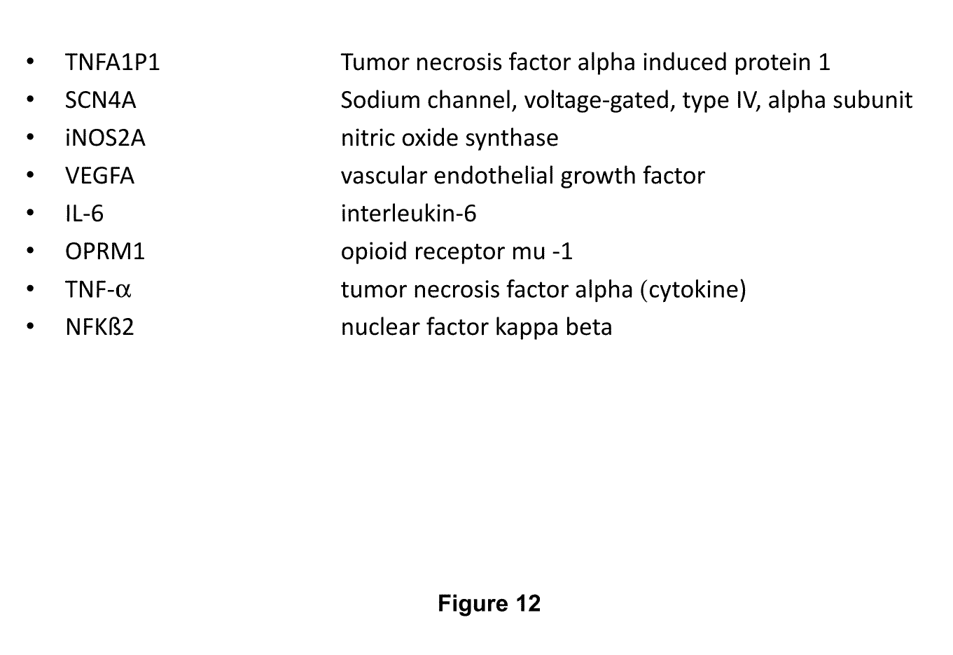

FIG. 13 is a schematic depicting the experimental design for miRNA target validation.

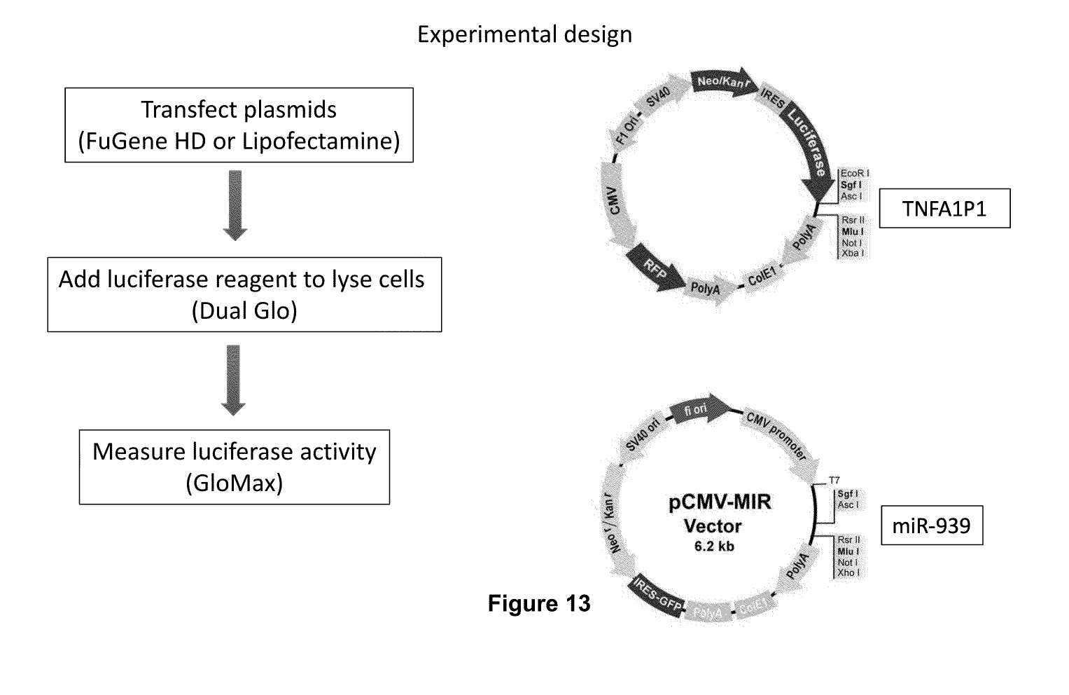

FIG. 14 depicts the results of a mir939 target validation experiment.

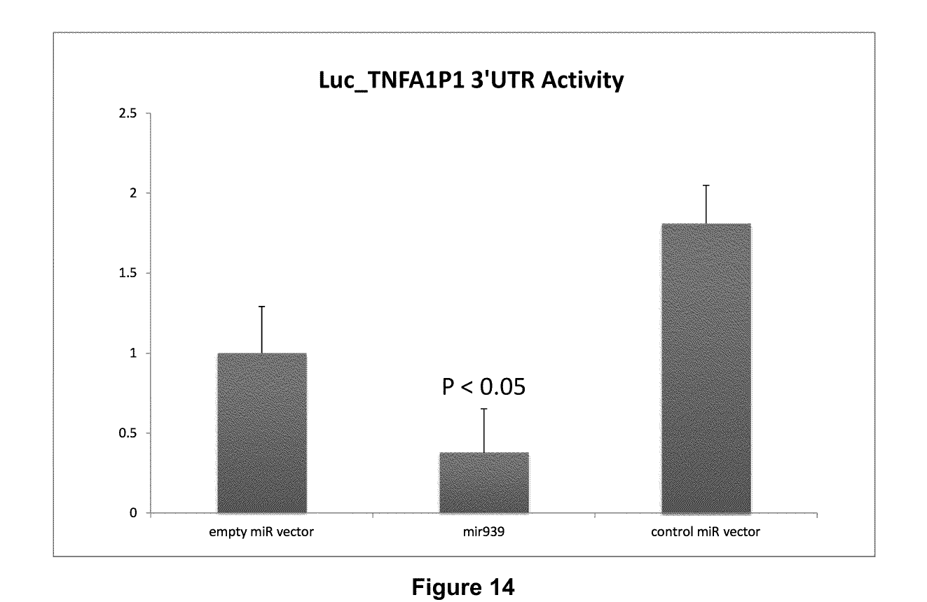

FIG. 15 depicts the results of a mir939 target validation experiment.

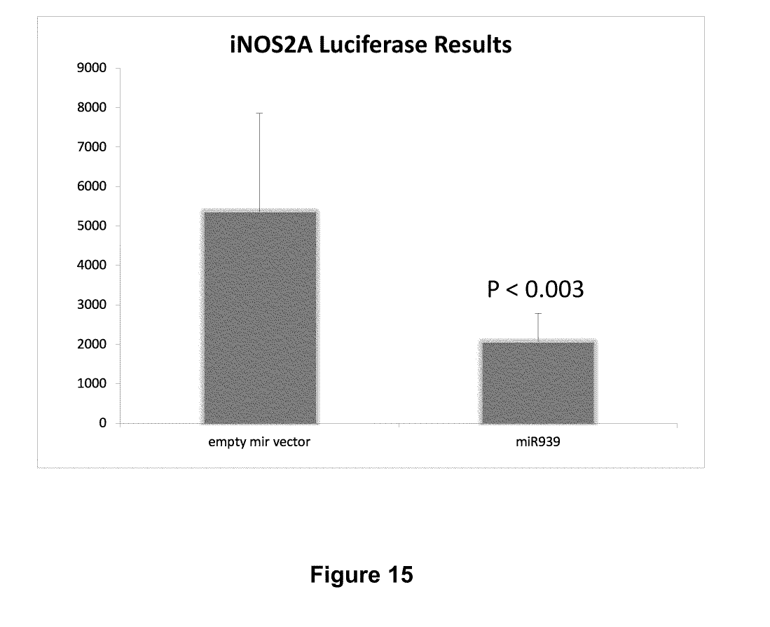

FIG. 16 depicts the results of a mir939 target validation experiment.

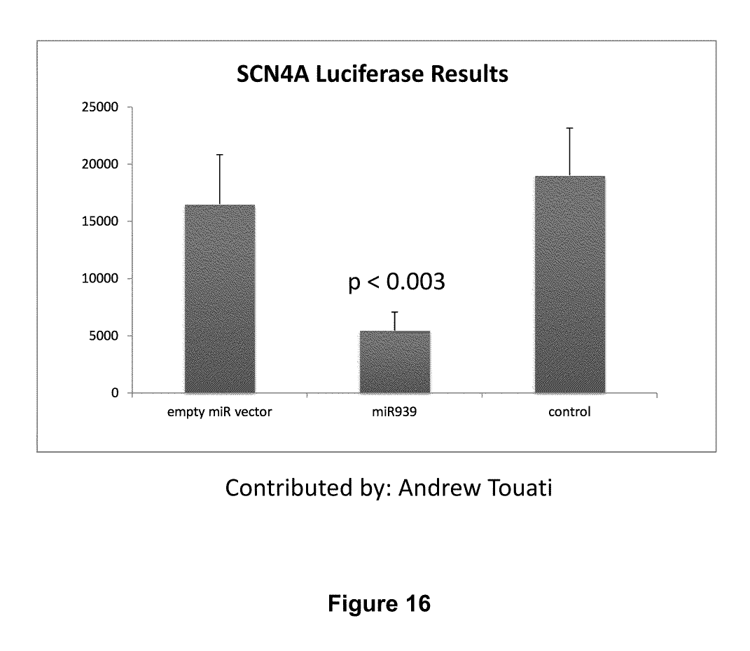

FIG. 17 depicts the results of a mir939 target validation experiment.

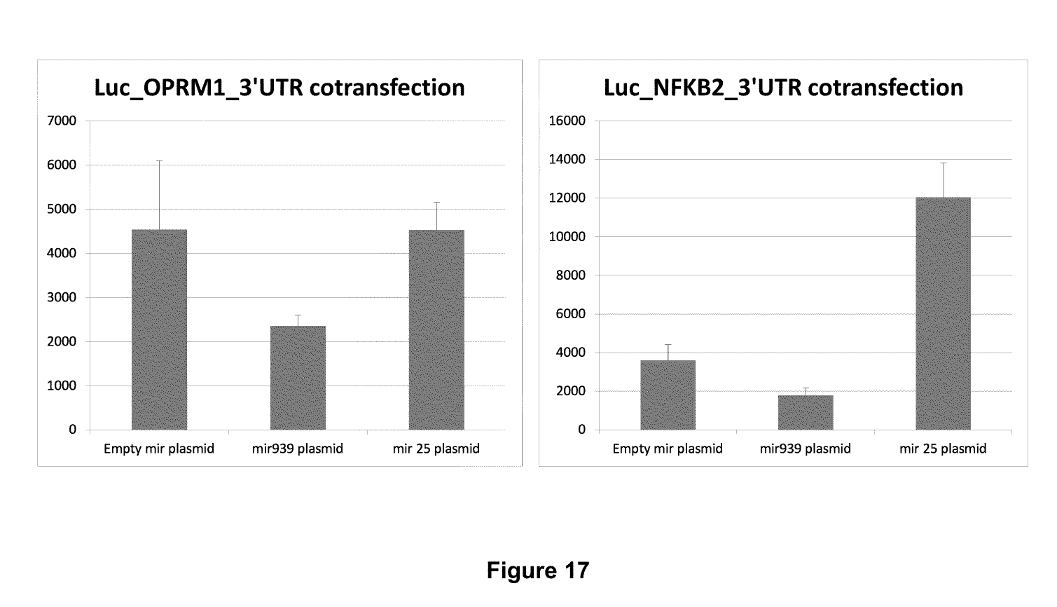

### DETAILED DESCRIPTION

The present invention relates to the discovery that the expression levels of some microRNAs (miRNAs) and small nucleolar RNAs (snoRNAs) are associated with neuropathic pain, including complex regional pain syndrome. Thus, in various embodiments described herein, the methods of the invention relate to methods of diagnosing a subject as having neuropathic pain, methods of assessing a subject's risk of having or developing neuropathic pain, methods of assessing the severity of a subject's neuropathic pain, and methods of stratifying a subject having neuropathic pain for assignment in a clinical trial. In some embodiments, a miRNAs or snoRNAs associated with neuropathic pain are up-regulated, while in other embodiments, the miRNAs or snoRNAs associated with neuropathic pain is down-regulated. Thus, the invention relates to compositions and methods useful for the detection and quantification of miRNAs and snoRNAs for the diagnosis, assessment, and characterization of neuropathic pain in a subject in need thereof, based upon the expression level of at least one miRNA or snoRNA that is associated with neuropathic pain.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detachable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antisense,” as used herein, refers to a nucleic acid sequence which is complementary to a target sequence, such as, by way of example, complementary to a target miRNA or snoRNA sequence, including, but not limited to, a mature target miRNA sequence, or a sub-sequence thereof. Typically, an antisense sequence is fully complementary to the target sequence across the full length of the antisense nucleic acid sequence.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60%, and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “dysregulated” and “dysregulation” as used herein describes a decreased (down-regulated) or increased (up-regulated) level of expression of a miRNA or snoRNA present and detected in a sample obtained from subject as compared to the level of expression of that miRNA or snoRNA present in a comparator sample, such as a comparator sample obtained from one or more normal, not-at-risk subjects, or from the same subject at a different time point. In some instances, the level of miRNA or snoRNA expression is compared with an average value obtained from more than one not-at-risk individuals. In other instances, the level of miRNA or snoRNA expression is compared with a miRNA level or snoRNA level assessed in a sample obtained from one normal, not-at-risk subject.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a comparator.

“Differentially decreased express” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a comparator.

As used herein “endogenous” refers to any material from or produced inside an organism, cell, tissue or system.

The term “expression” as used herein is defined as the transcription and/or translation of a particular nucleotide sequence.

“Fragment” as the term is used herein, is a nucleic acid sequence that differs in length (i.e., in the number of nucleotides) from the length of a reference nucleic acid sequence, but retains essential properties of the reference molecule. Preferably, the fragment is at least about 50% of the length of the reference nucleic acid sequence. More preferably, the fragment is at least about 75% of the length of the reference nucleic acid sequence. Even more preferably, the fragment is at least about 95% of the length of the reference nucleic acid sequence.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

AS used herein, “homology” is used synonymously with “identity.”

As used herein, “hybridization,” “hybridize(s)” or “capable of hybridizing” is understood to mean the forming of a double or triple stranded molecule or a molecule with partial double or triple stranded nature. Complementary sequences in the nucleic acids pair with each other to form a double helix. The resulting double-stranded nucleic acid is a “hybrid.” Hybridization may be between, for example two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids (e.g., DNA compounds). One or both of the nucleic acids may be immobilized on a solid support. Hybridization techniques may be used to detect and isolate specific sequences, measure homology, or define other characteristics of one or both strands. The stability of a hybrid depends on a variety of factors including the length of complementarity, the presence of mismatches within the complementary region, the temperature and the concentration of salt in the reaction or nucleotide modifications in one of the two strands of the hybrid. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for probe hybridizations. In a particularly preferred embodiment, huybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

As used herein, an “instructional material” includes a publication, a recording, a diagram, or any other medium of expression which can be used to communicate the usefulness of a compound, composition, vector, method or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gen messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intestinally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nuclei acids composed of bases other than the five biologically occurring bases (adenine, guanine, thymine, cystosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gen, including but not limited to deletion, insertion, substitution of one or more nucleotides, or fusion to other polynucleotide sequences.

As used herein, the term “promoter/regulatory sequence” means a nucleic acid sequence which is required for expression of a gen product operably linked to the promoter/regulator sequence. In some instances, this sequence may be the core promoter sequence and in other instances, this sequence may also include an enhancer sequence and other regulatory elements which are required for expression of the gen product. The promoter/regulatory sequence may, for example, be one which expresses the gene product in an inducible manner.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

The term “recombinant DNA” as used herein is defined as DNA produced by joining pieces of DNA from difference sources.

The term “recombinant polypeptide” as used herein is defined as a polypeptide produced by using recombinant DNA methods.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Synthetic mutant” includes any purposefully generated mutant or variant protein or nucleic acid. Such mutants can be generated by, for example, chemical mutagenesis, polymerase chain reaction (PCR) based approached, or primer-based mutagenesis strategies well known to those skilled in the art.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by the invention include, but are not restricted to, oligonucleotides, nucleic acids, antibodies, cell membrane receptors, monoclonal antibodies and antisera reactive with specific antigenic determinants (such as on viruses, cells or other materials), drugs, peptides, cofactors, lectins, sugars, polysaccharides, cells, cellular membranes, and organelles. Targets are sometimes referred to in the art as anti-probes.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in may regions, identical. A variant and reference peptide can differ in amino acid sequence by one or more substitutions, additions, deletions in any combination. A variant of a nucleic acid or peptide can be naturally occurring such as an allelic variant, or can be variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3 4, 5, 5.3, and 6. This applies regardless of the breadth of the range.

### Description

The present invention relates to the discovery that the level of expression of particular microRNAs (miRNAs) and small nucleolar RNAs (snoRNAs) is associated with neuropathic pain. In some embodiments, a miRNA or snoRNA associated with neuropathic pain is up-regulated, or expressed at a higher than normal level. In other embodiments, a miRNA or snoRNA associated with neuropathic pain is down-regulated, or expressed at a lower than normal level. Thus, the invention relates to compositions and methods useful for the diagnosis, assessment, and characterization of neuropathic pain in a subject in need thereof, based upon the expression level of at least one miRNA or snoRNA that is associated with neuropathic pain.

In various embodiments, the methods of the invention relate to methods of assessing a subject's risk of having or developing neuropathic pain, methods of assessing the severity of a subject's neuropathic pain, methods of diagnosing neuropathic pain, methods of characterizing neuropathic pain, and methods of stratifying a subject having the neuropathic pain in a clinical trial.

In a specific embodiment, the neuropathic pain is complex regional pain syndrome (CPRS). In various embodiments of the compositions and methods of the invention described herein, the miRNA or snoRNA associated with neuropathic pain is at least one of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

### Assays

The present invention relates to the discovery that the expression level of particular miRNAs and snoRNAs is associated with the presence, development, progression and severity of neuropathic pain. In various embodiments, the invention relates to a genetic screening assay of a subject to determine the level of expression of at least one miRNA or snoRNA associated with neuropathic pain in the subject. The present invention provides methods of assessing level of at least one miRNA or snoRNA associated with neuropathic pain, as well as methods of diagnosing a subject as having, or as being at risk of developing, neuropathic pain based upon the level of expression of at least one miRNA or snoRNA associated with neuropathic pain. In some embodiments, the diagnostic assays described herein are in vitro assays. In other embodiments, the diagnostic assays described herein are in vivo assays.

In one embodiment, the method of the invention is a diagnostic assay for assessing the presence, development, progression and severity of neuropathic pain in a subject in need thereof, by determining whether the level of at least one miRNA or snoRNA associated with neuropathic pain is increased in a biological sample obtained from the subject. In various embodiments, to determine whether the level of the at least one miRNA or snoRNA associated with neuropathic pain is increased in a biological sample obtained from the subject, the level of the at least one miRNA or snoRNA is compared with the level of at least one comparator control, such as a positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay. The miRNA or snoRNA identified by the assay can be any miRNA or snoRNA that is associated with neuropathic pain. In some embodiments, the miRNA or snoRNA is at least one of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In various embodiments of the invention, the at least one miRNA or snoRNA associated with neuropathic pain is at least two miRNAs or snoRNAs, at least three miRNAs or snoRNAs, at least four miRNAs or snoRNAs, at least five miRNAs or snoRNAs, at least six miRNAs or snoRNAs, at least seven miRNAs or snoRNAs, at least eight miRNAs or snoRNAs, at least nine miRNAs or snoRNAs, at least ten miRNAs or snoRNAs. The results of the diagnostic assay can be used alone, or in combination with other information from the subject, or other information from the biological sample obtained from the subject.

In another embodiment, the methods of the invention is an assay for monitoring the effectiveness of a treatment administered to a subject in need thereof, by determining whether the level of at least one miRNA or snoRNA associated with neuropathic pain is increased in a biological sample obtained from the subject. In various embodiments, to determine whether the level of the at least one miRNA or snoRNA associated with neuropathic pain is increased in a biological sample obtained from the subject, the level of the at least one miRNA or snoRNA is compared with the level of at least one comparator control, such as positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay. The miRNA or snoRNA identified by the assay can be any miRNA or snoRNA that is associated with neuropathic pain. In some embodiments, the miRNA or snoRNA is at least one of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In various embodiments of the invention, the at least one miRNA or snoRNA associated with neuropathic pain is at least two miRNAs or snoRNAs, at least three miRNAs or snoRNAs, at least four miRNAs or snoRNAs, at least five miRNAs or snoRNAs, at least six miRNAs or snoRNAs, at least seven miRNAs or snoRNAs, at least eight miRNAs or snoRNAs, at least nine miRNAs or snoRNAs, at least ten miRNAs or snoRNAs. The results of the diagnostic assay can be used alone, or in combination with other information from the subject, or other information from the biological sample obtained from the subject.

In a further embodiment, the method of the invention is an assay for assessing neuropathic pain in a subject for the purpose of stratifying the subject for assignment in a clinical trial, by determining whether the level of at least one miRNA or snoRNA associated with neuropathic pain is increased in a biological sample obtained from the subject. In various embodiments, to determine whether the level of the at least one miRNA or snoRNA associated with neuropathic pain is increased in a biological sample obtained from the subject, the level of the at least one miRNA or snoRNA is compared with the level of at least one comparator control, such as a positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay. The miRNA or snoRNA identified by the assay can be any miRNA or snoRNA that is associated with neuropathic pain. The subject can be stratified into a clinical trial based upon the information obtained from the assay, including, but not limited to, the severity of neuropathic pain, or the expression level of at least one miRNA or snoRNA associated with neuropathic pain. In some embodiments, the miRNA or snoRNA is at least one of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201. In various embodiments of the invention, the at least one miRNA or snoRNA associated with neuropathic pain is at least two miRNAs or snoRNAs, at least three miRNAs or snoRNAs, at least four miRNAs or snoRNAs, at least five miRNAs or snoRNAs, at least six miRNAs or snoRNAs, at least seven miRNAs or snoRNAs, at least eight miRNAs or snoRNAs, at least nine miRNAs or snoRNAs, at least ten miRNAs or snoRNAs. The results of the diagnostic assay can be used alone, or in combination with other information from the subject, or other information from the biological sample obtained from the subject.

In various embodiments of the assays of the invention, the level of the at least one miRNA or snoRNA associated with neuropathic pain is determined to be up-regulated when the level of the at least one miRNA or snoRNA is increased y at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In other various embodiments of the assays of the invention, the level of miRNA or snoRNA associated with neuropathic pain is determined to be down-regulated when the level of the at least one miRNA or snoRNA is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In the assay methods of the invention, a test biological sample from a subject is assessed for the expression level of at least one miRNA or snoRNA associated with neuropathic pain. The test biological sample can be an in vitro sample or an in vivo sample. In various embodiments, the subject is a human subject, and may be of any race, sex and age. Representative subjects include those who are suspected of having neuropathic pain, those who have been diagnosed with neuropathic pain, those who have neuropathic pain, those who have had neuropathic pain, those who at risk of a recurrence of neuropathic pain, and those who are at risk of developing neuropathic pain.

In some embodiments, a neuropathic pain associated miRNA-binding molecule is used in vivo for the diagnosis of neuropathic pain. In some embodiments, the neuropathic pain associated miRNA-binding molecule is nucleic acid that hybridizes with a neuropathic pain associated miRNA. In some embodiments, a neuropathic pain associated snoRNA-binding molecule is used in vivo for the diagnosis of neuropathic pain. In some embodiments, the neuropathic pain associated snoRNA-binding molecule is nucleic acid that hybridizes with a neuropathic pain associated snoRNA.

In one embodiment, the test sample is a sample containing at least a fragment of a nucleic acid comprising a miRNA associated with neuropathic pain. The term, “fragment,” as used herein, indicates that the portion of a nucleic acid (e.g., DNA, mRNA or cDNA) that is sufficient to identify it as comprising a miRNA associated with neuropathic pain.

In some embodiments, the test sample is prepared from a biological sample obtained from the subject. The biological sample can be a sample from any source which contains a nucleic acid comprising neuropathic pain associated miRNA, such as a body fluid (e.g., blood, plasma, serum, saliva, urine, etc.), or a tissue, or an exosome, or a cell, or a combination thereof. A biological sample can be obtained by appropriate methods, such as, by way of examples, biopsy or fluid draw. The biological sample can be used as the test sample; alternatively, the biological sample can be processed to enhance access to polypeptides, nucleic acids, or copies of nucleic acids (e.g., copies of nucleic acids comprising a miRNA associated with neuropathic pain), and the processed biological sample can then be used as the test sample. For example, in various embodiments, nucleic acid is prepared from a biological sample, for use in the methods. Alternatively or in addition, if desired, an amplification method can be used to amplify nucleic acids comprising all or a fragment of a nucleic acid in a biological sample, for use as the test sample in the assessment of the expression level of a miRNA associated with neuropathic pain.

The test sample is assessed to determine the level of expression of at least one miRNA or snoRNA associated with neuropathic pain present in the nucleic acid of the subject. In general, detecting a miRNA or snoRNA may be carried out by determining the presence or absence of a nucleic acid containing a miRNA or snoRNA of interest in the test sample.

In some embodiments, hybridization methods, such as Northern analysis, or in situ hybridizations, can be used (see Current Protocols in Molecular Biology, 2012, Ausubel, F. et al., eds., John Wiley & Sons, including all supplements). For example, the presence of a miRNA associated with neuropathic pain can be indicated by hybridization to a nucleic acid probe. A “nucleic acid probe,” as used herein, can be a nucleic acid probe, such as a DNA probe or an RNA probe. For representative examples of use of nucleic acid probes, see, for example, U.S. Pat. Nos. 5,288,611 and 4,851,330.

To detect at least one miRNA or snoRNA of interest, a hybridization sample is formed by contacting the test sample with at least one nucleic acid probe. A preferred probe for detecting miRNA is a labeled nucleic acid probe capable of hybridizing to miRNA. The nucleic acid probe can be, for example, a full-length nucleic acid molecule, or a portion thereof, such as an oligonucleotide of at least 10, 15, or 25 nucleotides in length and sufficient to specifically hybridize under stringent conditions to appropriate miRNA. The hybridization sample is maintained under conditions which are sufficient to allow specific hybridization of the nucleic acid probe to a miRNA target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a miRNA in the test sample, the sequence that is present in the nucleic acid probe is also present in the miRNA of the subject. More than one nucleic acid probe can also be used concurrently in this method. Specific hybridization of any one of the nucleic acid probes is indicative of the presence of the miRNA of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising at least one miRNA or snoRNA of interest. Hybridization of the PNA probe to a nucleic acid sequence is indicative of the presence of a miRNA of interest.

Direct sequence analysis can also be used to defect miRNAs of interest. A sample comprising nucleic acid can be used, and PCR or other appropriate methods can be used to amplify all or a fragment of the nucleic acid, and/or its flanking sequences, if desired.

In another embodiment, arrays of oligonucleotide probes that are complementary to target nucleic acid sequences from a subject can be used to detect, identify and quantify miRNAs associated with neuropathic pain. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fador et al., Science, 251:767-777 (1991), Pirrung et el., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a sample containing miRNA is hybridized with the array and scanned for miRNAs. Hybridization and scanning are generally carried out by methods described herein and also in, e.g., Published PCT Application Nos. WO 92/10092 and WO 95/11995. and U.S. Pat. No. 5,424,186, the entire teachings of which are incorporated by reference herein.

In brief, a target miRNA sequence is amplified by well-known amplification techniques, e.g., RT, PCR. Typically, this involves the use of primer sequences that are complementary to the target miRNA. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect miRNAs of interest. Representative methods include direct manual sequencing (1988, Church and Gilbert. Proc. Natl. Acad. Sci. USA 81:1991-1995; 1997, Sanger et al., Proc. Natl. Acad. Sci. 74:5463:5467; Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., 1981, Proc. Natl. Acad. Sci. USA 86:232-236), mobility shift analysis (Orita et al., 1989, Proc. Natl. Acad. Sci. USA 86:2766-2770; Rosenbaum and Reissner, 1987, Biophys. Chem. 265:1275, 1991, Keen et al., Trends Genet. 7:5); RNase protection assays (Myers, et al., 1985, Science 230:1242); Luminex xMAP™ technology; high-throuput sequencing (HTS) (Gundry and Vijg, 2011, Mutat Res, doi:10.1016/j.mrfmmm.2011, 10.001); next-generation sequencing (NGS) (Voelkerding et al., 2009, Clinical Chemistry 55:641-658; Su et al., 2011, Expert Rev Mol Diagn. 11:333-343; Ji and Myllykaagas, 2001, Biotechnol Genet Eng Rev 27:135:158); and/or ion semiconductor sequencing (Rusk, 2011, Nature Methods doi: 10.1038/nmeth.f.330; Rothberg et al., 2011, Nature 475:348-352). These and other methods, alone or in combination, can be used to detect and quantity of at least one miRNA or snoRNA of interest, in a biological sample obtained from a subject. In one embodiment of the invention, the methods of assessing a biological sample to detect and quantify a miRNA of interest, as described herein, are used to diagnose, assess and characterize neuropathic pain in a subject in need thereof.

The probes and primers according to the invention can be labeled directly or indirectly with a radioactive or nonradioactive compound, by methods well known to those skilled in the art, in order to obtain a detachable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes and luminescent agents such as radioluminescent, chemoluminescent, bioluminescent, fluorescent or phosphorescent agents.

Nucleic acids can be obtained from the biological sample using known techniques. Nucleic acid herein includes RNA, including mRNA, miRNA, etc. The nucleic acid can be double-stranded or single-stranded (i.e., a sense or an antisense single strand) and can be complementary to a nucleic acid encoding a polypeptide. The nucleic acid content may also be obtained from an extraction performed on a fresh or fixed biological sample.

There are many methods known in the art for the detection of specific nucleic acid sequences and new methods are continually reported. A great majority of the known specific nucleic acid detection methods utilize nucleic acid probes in specific hybridization reactions.

In the Northern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. Nos. 6,519,693 and 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Northern blotting, levels of the polymorphic nucleic acid can be compared to wild-type levels of the nucleic acid.

A further process for the detection of hybridized nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

Nucleic acid amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

Stem-loop RT-PCR is a PCR method that is useful in the methods of the invention to amplify and quantify miRNAs of interest (See Caifu et al., 2005, Nucleic Acids Research 33:e179; Mestdagh et al., 2008, Nucleic Acids Research 36:e143; Varkonyi-Gasic et al., 2011, Methods Mol Biol. 744:145-57). Briefly, the method includes two steps: RT and real-time PCR. First, a stem-loop RT primer is transcriptase. The, the RT products are quantified using conventional real-time PCR.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the template nucleic acid under conditions of stringency that prevent non-specific binding but permit binding of this template nucleic acid which has a significant level of homology with the probe or primer.

Among the conditions of stringency is the melting temperature (Tm) for the amplification step using the set of primers, which is in the range of about 50° C. to about 95° C. Typical hybridization and washing stringency conditions depend in part on the size (i.e., number of nucleotides in length) of the template nucleic acid or the oligonucleotide probe, the base composition and monovalent and divalent cation concentrations (Ausubel et al., 1994, eds Current Protocols in Molecular Biology).

In a preferred embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 50° C. to 95° C. Preferably, the Tm for any one of the hydrolysis-probes of the present invention is in the range of about 55° C. to about 80° C. Most preferably, the Tm applied for any one of the hydrolysis-probes of the present invention is about 75° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a nucleic acid sequence of the miRNA of interest, and more particularly the primer includes 12 or more contiguous nucleotides substantially complementary to the sequence of the miRNA or snoRNA of interest. Preferably, a primer featured in the invention includes a nucleotide sequence sufficiently complementary to hybridize to a nucleic acid sequence of about 12 to 25 nucleotides. More preferably, the primer differs by no more than 1, 2 or 3 nucleotides from the target nucleotide sequence. In another aspect, the length of the primer can vary in length, preferably about 15 to 28 nucleotides in length (e.g., 15, 16, 17, 18, 19, 20, 21 , 22, 23, 24, 25, 26, 27, or 28 nucleotides in length).

### Kits

The present invention also pertains to kits useful in the methods of the invention. Such kits comprise components useful in any of the methods described herein, including for example, hybridization probes or primers (e.g., labeled probes or primers), reagents for detection of labeled molecules, oligonucleotide arrays, restriction enzymes, antibodies, allele-specific oligonucleotides, means for amplification of a subject's nucleic acids, means for reverse transcribing a subject's RNA, means for analyzing a subject's nucleic acid sequence, and instructional materials. For example, in one embodiment, the kit comprises components useful for the detection and quantification of at least one miRNA or snoRNA associated with neuropathic pain. In a preferred embodiment of the invention, the kit comprises components for detecting one or more of the miRNAs associated with neuropathic pain as elsewhere described herein.

### EXPERIMENTAL EXAMPLES

The invention is further described in detail by reference to the following experiment examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### MicroRNA Modulation in Complex Regional Pain Syndrome

Aberrant expression of small noncoding RNAs called microRNAs (miRNAs) is a feature of some human diseases. An objective of the studies described herein was to identify miRNA modulation inpatients with complex regional pain syndrome (CRPS) a chronic pain condition resulting from dysfunction in the central and/or peripheral nervous systems. Due to a multitude of inciting pathologies, symptoms and treatment conditions, the CRPS patient population is very heterogeneous. A goal of the studies described herein was to identify differentially expressed miRNAs in blood and explore their utility in patient stratification. miRNA profiles can be useful in patient stratification and have utility as potential biomarkers for pain. Differentially expressed miRNAs can provide molecular insights into gene regulation and lend insight to new therapeutic intervention strategies for CRPS.

The differential expression of 18 miRNAs in whole blood from patients with CRPS were observed and compared to control samples. Thus, multiple miRNAs were significantly different between patients and control subjects as compared with three inflammatory and immune related markers. As described herein, the clustering of 60% of patients with CRPS on the basis of a miRNA profile demonstrates that clinically relevant stratification of the patient population is possible on the basis of alterations in miRNA expression. miRNAs recognize their target mRNAs using the 2-8 nucleotide sequence at the 5′ region of the miRNA called the seed sequence. Target prediction algorithms use different parameters to provide candidate target genes for miRNAs (Sethapathy et al., 2006, Nat. Methods 3:881-886). Earlier success with TargetScan (von Schack et al., 2011, PLoS One 6:c17670) led to the use of TargetScan (Lewis et al., 2005, Cell 120:15-20) to perform the initial analysis for miRNAs identified to be differentially expressed in CRPS. Bioinformatic prediction of the significantly altered miRNAs showed that these miRNAs can potentially modulate mRNAs of a number of genes relevant in CRPS including inflammatory mediators, ion channels, and G protein-coupled receptors. For example, a bioinformatics-based prediction indicates that hsa-miR-939 can target vascular endothelial growth factor A (VEGF A), inducible nitric oxide synthase 2A, and the alpha subunit of voltage-gated sodium channel type IV, and that hsa-miR-25 can target endothelin receptor type B. Since one of the predicted gene targets for hsa-miR-939 is VEGF A, the upregulation of VEGF in the serum of CRPS patients is consistent with this prediction.

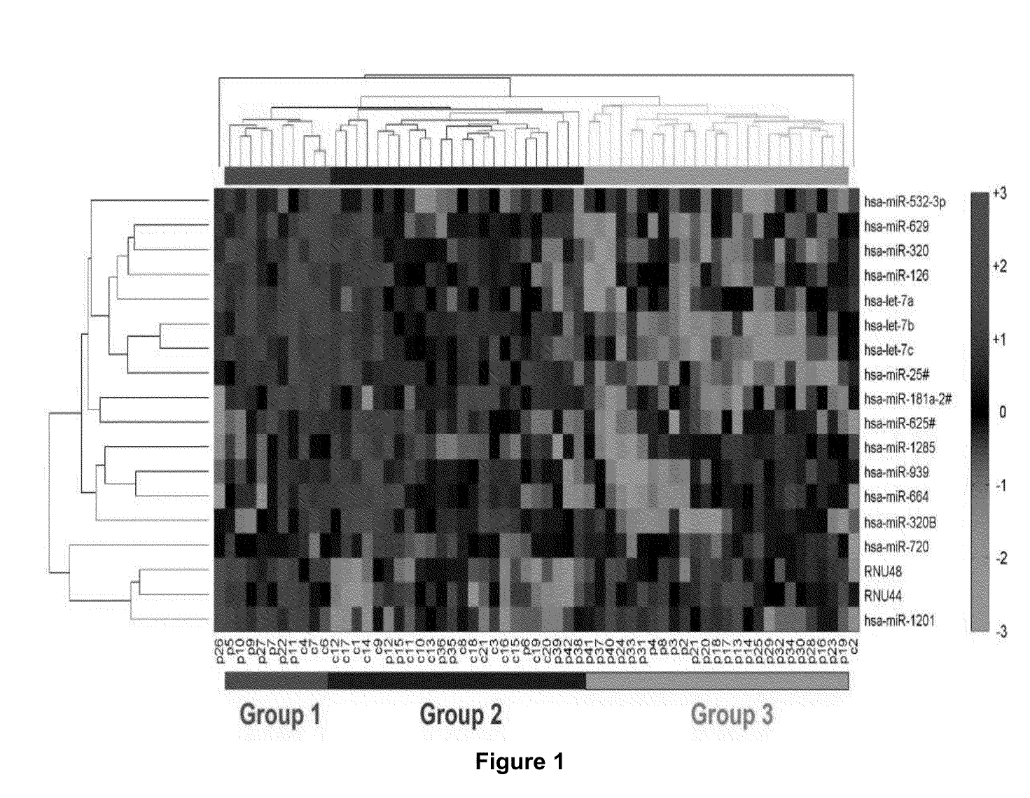

The miRNAs shown in FIG. 1 from the CRPS study were compared with miRNAs altered in some of the rodent models for pain that were investigated. While there was no overlap in the miRNAs identified from studies that focused on a limited number of miRNAs, (Bai et al., 2007, Mol. Pain 3:15; Kusuka et al., 2011, Mol. Pain 7:17; Poh et al., 2011, Eur. J. Pain 15:801, e801-812, Aldrich et al., 2009, Neuroscience 164:711-723; Favereaux et al., 2011, EMBO J. 30:3830-3841) profiling from dorsal root ganglion (DRG) from the rat spinal nerve ligation (SNL) model (von Schack et al., 2011, PLoS One 6:e17670), described elsewhere herein, showed that the expression of four miRNAs hsa-miR-126, hsa-let-7a, hsa-let-7b and hsa-let-7c (multiple copies of let-7 family of miRNAs are present in the genome; isoforms are distinguished by a letter placed after let-7 to indicate a slightly different sequence) was significantly altered in both CRPS blood and rat DRG after SNL. Profiling of 369 miRNAs was performed in rat DRG 4 weeks post SNL (von Schack et al., 2011, PLoS One 6:e17670), whereas the current study profiled 758 miRNAs. Thus, not all miRNAs identified in the present CRPS study have been profiled in rat DRG. This finding is an indication of the potential translational value that can be achieved from miRNA profiling.

Expression of small nucleolar RNAs (snoRNAs) RNU44 and RNU48 was found to be altered in CRPS patients. RNU44 and RNU48 have been widely used for miRNA data normalization, not a recent study reported that normalizing miRNA expression data to these recommended snoRNAs introduced bias in associations between miRNA and pathology or outcome (Gee et al., 2001, Br. J. Cancer 104:1168-1177).

It is described herein that hsa-mir-150 correlates with headache and several other miRNA correlations with comorbidities such as high blood pressure, thyroid disorder, use of medications including narcotics and antiepileptic drugs. These results indicate the broader utility of performing miRNA profiling and could provide additional molecular insights into disease biology occurring as comorbidities or use of specific medications.

The studies described herein indicate that miRNA profiling can serve as a novel approach for patient stratification. Stratification based on the miRNA profile resulted in identification of additional markers that were not significant when all CRPS patients were analyzed as a single group. The potential for identifying several miRNAs as signatures rather than relying on one specific biomarker will increase the chances of successful treatment in the heterogeneous CRPS patient population. Identifying informative benchmarks will be an exceptionally valuable tool for assisting physicians in choosing treatment options and for stratifying patients in clinical trials.

Although not wishing to be bound by any particular theory, the ability to cluster 60% of the patients with CRPS suggest that miRNA profiling can serve as a novel, clinically relevant approach for patient stratification. Grouping patients based on miRNA profiling resulted in the identification of additional markers that did not appear significant when the whole CRPS population was considered. This could be due to the etiology as well as the multitude of pathologies and symptoms associated with CRPS.

The materials and methods employed in this example are now described.

### miRNA

miRNAs in whole blood from 41 patients with CRPS and 20 controls were profiled using TaqMan low density array cards. Since neurogenic inflammation is known to play a significant role in CRPS, inflammatory markers were measured including chemokines, cytokines, and their soluble receptors in blood from the same individuals. Correlation analyses were performed for miRNAs, inflammatory markers and other parameters including disease symptoms, medication, and comorbid conditions.

### Standard Protocol Approvals, Registrations, and Patient Consents

All subjects were enrolled after giving informed consent.

### Inclusion and Exclusion Criteria

Patients with CRPS were recruited from the pain clinic of Drexel University College of Medicine and fulfilled the International Association for the Study of Pain (IASP) diagnostic criteria for CRPS (Harden et al., 2007, Pain Med. 8:326-331). Healthy control subjects were recruited from the general public. The exclusion criteria for all subjects included pregnancy, recent infection, lupus crythematosus, HIV/AIDS, rheumatoid arthritis, recent extracorporeal circulation (hemodialysis, bypass surgery, plasmapheresis), bone marrow transplant, immunosuppressive therapy, blood disorders (anemia, leukemia), thymectomy, or sarcoidosis.

### Patient Evaluation

All patients with CRPS received a complete neurological examination and pain evaluation (performed by RJS). Overall pain levels were determined on a 0-10 numerical rating scale (NRS) (0=no pain, 10=worst possible pain). Additional information collected included duration of CRPS in years, age of onset, and medications (narcotics, antiepileptics, antidepressants, and anxiolytics). The presence of other conditions such as radiculopathy, heart disease, arthritis, irritable bowel syndrome, high blood pressure, seizure disorder, spinal disk disease, high cholesterol, generalized anxiety disorder, depression, gastroesophageal reflux disease, migraines, and thyroid disease was also noted. Medical history and self-reported values for height and weight were obtained from normal healthy control subjects. Blood samples were collected from 41 patients while they were taking their current medications and from 20 controls.

### miRNA Profiling

Whole blood was collected in PAXgene blood RNA tubes (BD Biosciences, Bedford, Mass.) RNA was isolated using a PAX gene blood miRNA kit (Qiagen, Valencia, Calif.) following the manufacturer's protocol. RNA concentration was measured using Nanodrop 1000 (NanoDrop Technologies, Wilmington, Del.)

Taqman Low Density Array (TLDA) microfluidic cards version A and B (Applied biosystems, Foster City, Calif.) were used to profile miRNAs and the protocol recommended by the vendor was followed. 50 ng of total RNA was used in each reaction for cDNA synthesis using a TaqMan microRNA reverse transcription kit and human megaplex RT primers for Pool A and Pool B. Preamplification was done using TaqMan preamplification master mix and human megaplex preamplification primers corresponding to Pool a and Pool B. TLDA cards were assayed on ABI PRISM 7900 Sequence detector using universal thermal cycling conditions of 50° C. for 2 minutes, 95° C. for 10 minutes, then 40 cycles of 95° C. for 15 seconds and 60° C. for 1 minute. The threshold level for background detection in SDS software was manually set to 0.2

### Data Analysis

Quantile normalization was applied to the cycle threshold (CT) values. Samples with CT values 32 and above were treated as undetected as recommended by the vendor. Fold change was calculated from raw (CT values using the 2^{ΔΔCT} methods (Schmittgen et al., 2008, Nat. Protoc. 3:1101-1108). The mean of the CT values of the 10 miRNAs with the lowest standard deviation was used as the endogeneous control in the calculation of ΔCT. Statistical significance of differences in ΔCT values between CRPS patients and controls was calculated by a 2-tailed independent samples t-test. The Benjamini-Hochberg false discovery rate correction (Hochberg et al., 1995, Journal of the Royal Statistical Society 57:289-300) was applied to the p-values. Pairwise Spearman correlation was calculated between various clinical markers and miRNAs. Hierarchical clustering of miRNAs and samples was performed along with the generation of a heatmap of miRNA expression. The samples were clustered into three groups of the basis of their miRNA expression levels and the correlations of other variables against these three groups were calculated.

### Determination of Cytokines/Chemokines and Their Soluble Receptors

The plasma was separated by centrifugation (3000 g for 15 minutes at 4° C.), split into 250 μL aliquots and stored at −70° C. The Milliplex Map high sensitivity 10 plex human cytokine kit (Millipore, Billerica, Mass.) was used to determine plasma levels of the following cytokines: interferon-gamma (IFNγ); the interleukins IL-1β, IL-2, IL-4, IL-5, IL-6, IL-7, IL-8, and IL-10; and tumor necrosis factor alpha (TNFα). The Milliplex Map human soluble cytokine receptor panel (Millipore, Billerica, Mass.) was used to determine the following soluble receptors: soluble glycoprotein 130 (sgp130) (a subunit of the IL-6 receptor complex); the TNFα soluble receptors sTNFRI and sTNFRII; and sRAGE, the soluble receptor for advanced glycation end products (AGEs). The plasma levels of the interleukin-1 receptor antagonist (IL1Ra) and the chemokine monocyte chemotactic protein-1 (MCP1) were determined with the Fluorokine MAP Multiplex Human Cytokine Panel A (R&D Systems, Minneapolis, Minn.). Assay results were determined on a Luminex-200 (Luminex, Austin, Tex.).

The results of the experiments are now described.

### miRNA Profiling

Three difference groups emerged from miRNA profiling. One group was comprised of 60% of CRPS patients and contained no control subjects. miRNA profiles from the remaining patients were interspersed among control samples in the other two groups. The differential expression of 18 miRNAs in CRPS patients was indentified. Analysis of inflammatory markers showed that vascular endothelial growth factor (VEGF), interleukin1 receptor antagonist (IL1Ra) and monocyte chemotactic protein-1 (MCP1) were significantly elevated in CRPS patients. VEGF and IL1Ra showed significant correlation with the patients reported pain levels. Analysis of the patients who were clustered according to their miRNA profile revealed correlations that were not significant in the total patient population. Correlation analysis of miRNAs detected in blood with additional parameters identified miRNAs associated with comorbidities such as headache, thyroid disorder and use of narcotics and antiepileptic drugs.

The average age of controls and patients was 42±12.7 and 45±11.7 years, respectively. The female to male ratio was 14/6 for controls and 27/14 for patients with CRPS. Average body mass index (BMI) for controls and patients was 24.12 and 28.58 respectively indicating normal BMI for control and obesity in the CRPS patient population.

### miRNA Alterations in CRPS

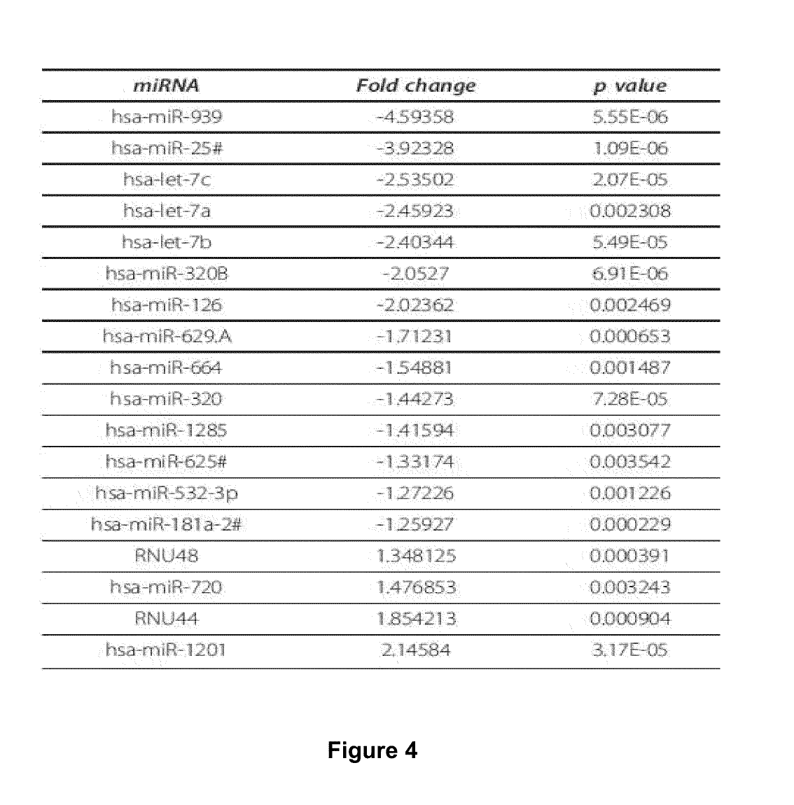

Two-failed t-tests were used to identify differential expression of miRNAs between patients and control samples. The fold changes and p values of significantly altered miRNAs are shown in FIG. 4. A clustergram of the samples and miRNAs identifying sample subpopulations with respect to the significant differentially expressed miRNAs is shown in FIG. 1 as a heat map of the log-transformed fold changes. Heat maps are commonly used for visualization of high-dimensional data in a two-dimensional image with colors representing the intensity values. It is typically used in gene expression analysis to represent the level of expression of many genes across a number of comparable samples. Clustering based on the fold change of significant miRNAs resulted in a clear separation of patient-only population from the rest of the subjects. A total of 60% of the patients (the right half of clustering) showed differential regulation for these miRNAs (mostly down regulation), whereas the rest of the patients were more heterogeneous and were clustered together with the control samples which showed considerable variability.

### Inflammatory Markers

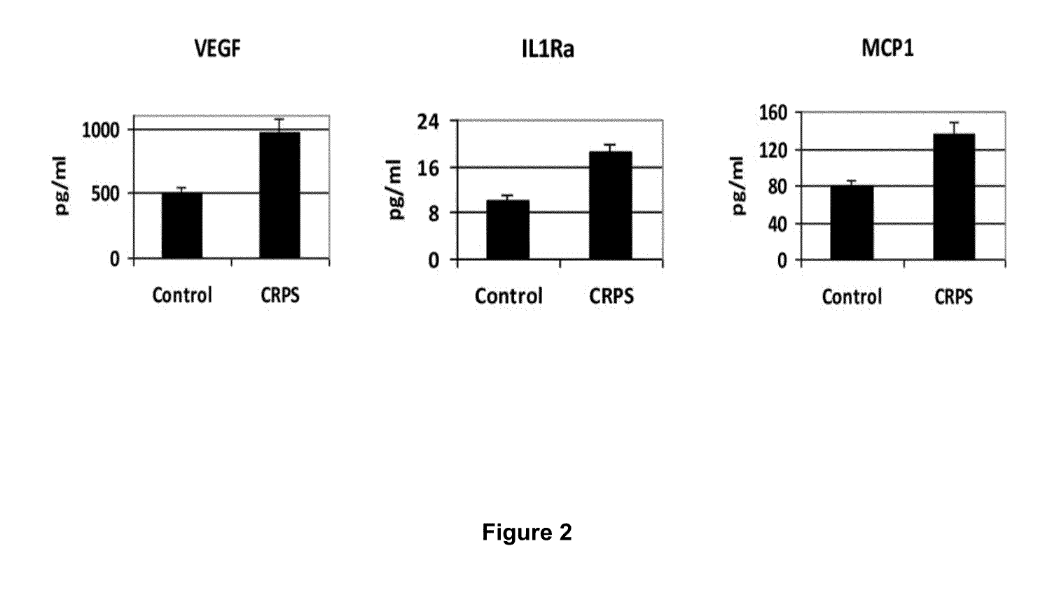

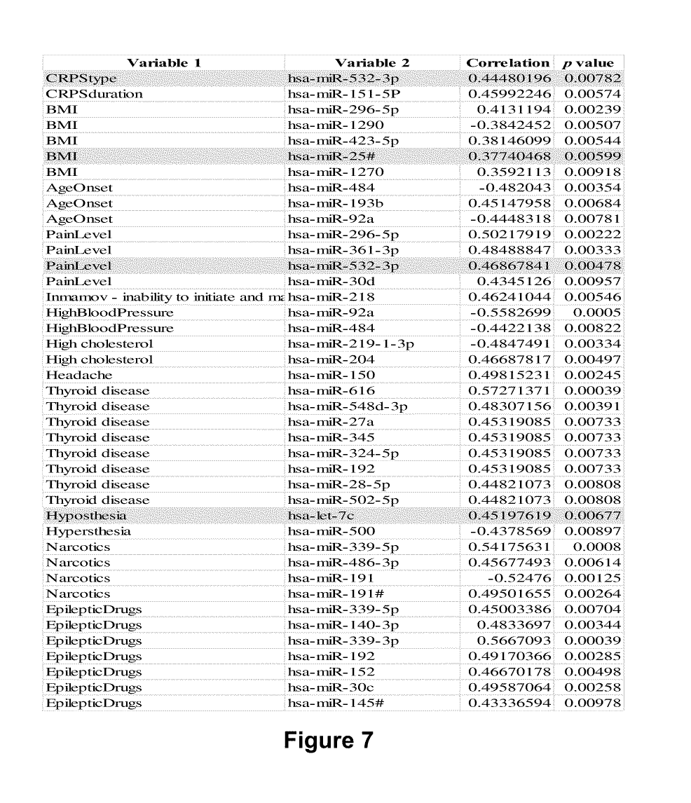

Analysis of inflammatory markers including chemokines, cytokines and their soluble receptors in blood from the same individuals showed changes in several markers (FIG. 7). Though there were trends, t-test (Benjamini-Hochberg adjustment of p values for multiple comparisons) showed that only VEGF, ILR1a and MCP1 levels were significantly elevated in CRPS patients (FIG. 2). VEGF and ILR1a were significantly correlated with the reported pain levels of the patients. VEGF also had a significant correlation with BMI (FIG. 7).

### Analysis of Subset of Patients with CRPS

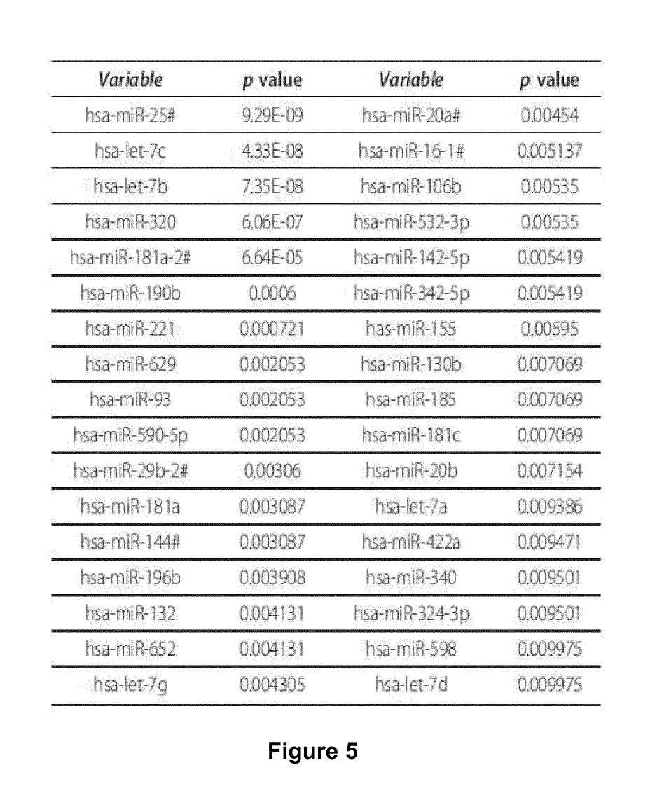

Since 60% of the CRPS patients segregated based on miRNA profile (FIG. 1 group 3), this subgroup of patients was analyzed further. Additional miRNAs that were identified to be significantly altered are shown in FIG. 5. For inflammatory and immune related markers, correlations of significance were observed for IL-6, IL-8, IL1Ra, MCP1 and VEGF when considering all CRPS patients versus control samples. Comparing subpopulation group 3 against other samples identified additional correlations for IL-4, IL-5, and TNFalpha. However IL-6, IL-8 and MCP1 were not significant in group 3. Thus, stratifying the patient population on the basis of the miRNA profile resulted in changes, predominantly toward the identification of additional indicators that could be clinically relevant in CRPS.

### Correlation Analysis

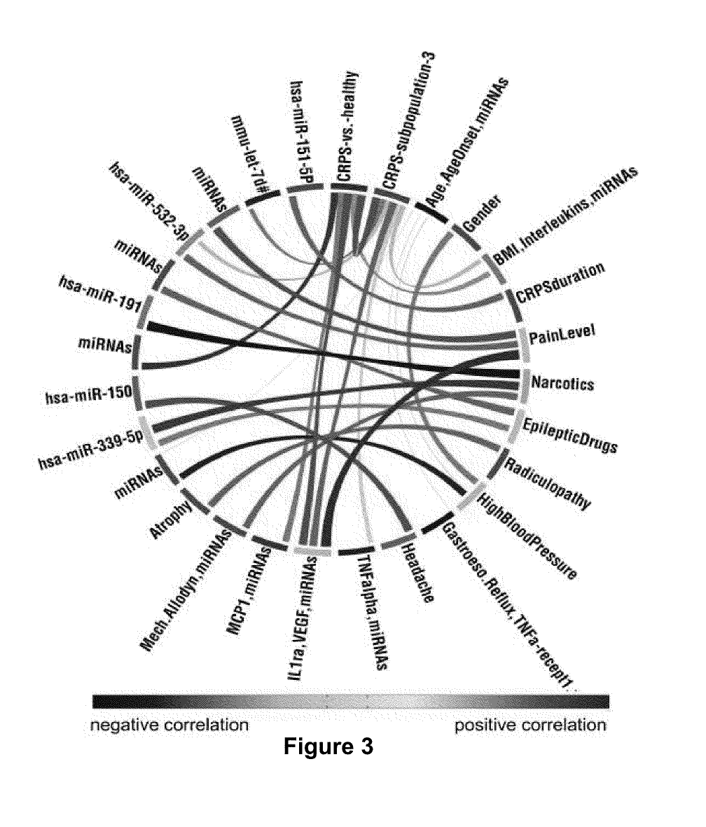

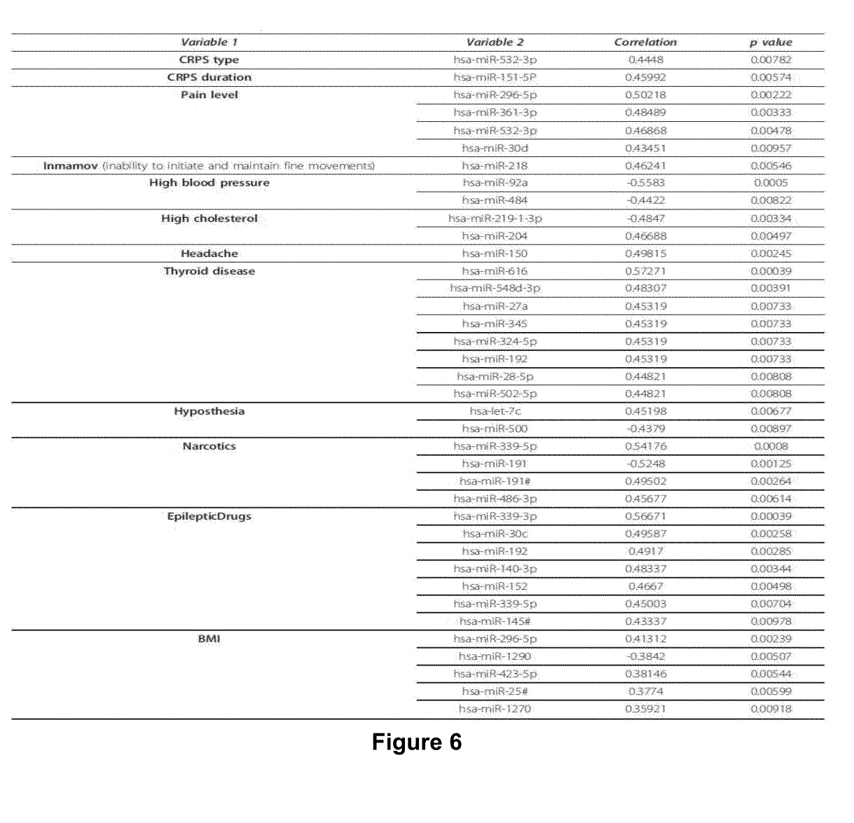

Additional correlation analyses were performed of all miRNAs detected in whole blood with other parameters including comorbidities. In addition to being in the list of 18 miRNAs identified to be differentially expressed in patients with CRPS, hsa-miR-532-3p was associated with CRPS type, pain level, IL1Ra, and VEGF (FIGS. 6 and 7). CPRS Type-2 patients from this study had higher hsa-miR-532 and higher VEGF levels compared to CRPS Type-1 patients. A strong correlation between miRNAs and comorbidities was observed, such as high blood pressure, cholesterol, thyroid disease, and use of narcotics and antiepileptic medications (FIGS. 6 and 7). These miRNAs did not overlap with the miRNAs that were modulated in CRPS (FIG. 4). Though not the primary objective, these results identified miRNA alterations that may be specific to comorbid conditions observed in patients with CRPS. A Circos diagram (Krzywinski et al., 2009, Genome Res. 19:1639-1645) capturing the significant correlations among all parameters analyzed are shown in FIG. 3.

### Example 2

### Exosome-Mediated miRNA Transfer in Pain

Complex regional pain syndrome (CRPS) is a disabling chronic neuropathic pain syndrome. The differential expression of 18 microRNAs (miRNAs) was identified in whole blood from CRPS patients indicating miRNA modulation in chronic pain and its potential utility in patient stratification (Orlova et al., 2011, J. Transl. Med. 9:195), as described elsewhere herein. The studies described herein establish that one or more of the miRNAs having altered expression in CRPS patients plays a role in inflammatory pain that is mediated via exosomes.

CRPS is a chronic neuropathic pain condition with a broad spectrum of symptoms including pain, inflammation, sensory dysfunction, impaired motor function, and trophic disturbances.

As described herein, the identification of exosome mediated transfer of nucleic acids in blood from CRPS patients provides insight into the molecular mechanisms underlying this debilitating disorder.

The materials and methods used in this example are now described.

### Characterization of Exosomes

Exosomes obtained from HEK293 cells, human aortic endothelial cells (HAEC), human blood and mouse blood are subjected to RibonucleoproteinImmunoPrecipitation-sequencing (RIP-Seq), a technique in which immunoprecipitation (IP) of RNA-binding protein is coupled to reverse transcription and sequencing. IP is performed using argonaute (Ago) antibody. Multiple miRNAs and their mRNA targets have been identified using Ago IP3. Identification of miRNA and mRNA in the exosomes shed light of the “cargo” being transported to target cells and the role they may play in this novel intercellular communication (Thery et al., 2006, Curr. Protoc. Cell Biol. Chapter 3: Unit 3, 22).

### Characterization of Exosomes from CRPS Patients

Eleven of the eighteen miRNAs differentially expressed in CRPS patients have been reported to be present in exosomes (www.exocarts.org). This is confirmed using exosomes isolated from blood samples from CRPS and controls.

Exosomes obtained from CRPS patients are subjected to RibonucleoproteinImmunoPrecipitation-sequencing (RIP-Seq), a technique in which immunoprecipitation (IP) of an RNA-binding protein is coupled to reverse transcription and sequencing. IP is performed using argonaute (Ago) antibody. Multiple miRNAs and their mRNA targets have been identified using Ago IP3. These studies enable direct identification of pulative mRNA targets for miRNAs and reduce the rate of false positive predictions commonly associated with bioinformatics predictions. Identification of miRNA and mRNA in the exosomes shed light on the “cargo” being transported to target cells and the role they may play in this novel intercellular communication (Thery et al., 2006, Curr. Protoc. Cell Biol. Chapter 3: Unit 3, 22).

### In vitro Validation of Alterations in Exosome miRNA in Response to Inflammatory Stimulus

RAW264.7 (mouse leukemic monocyte macrophage) and/or THP1 (human acute monocytic leukemia) are stimulated with lipolysaccharides (LPS). LPS from gram-negative bacteria which act as endotoxins and elicit strong immune responses. Exosomes are isolated and characterized from the cell culture supernatants and qPCR is performed for 18 human and 11 rodent miRNAs that are known to be differentially regulated in CRPS patients and in a mouse model of inflammatory pain, respectively.

### Exosome Mediated Information Transfer—Functional Confirmation

Neurogenic inflammation is common in CRPS. NFkappaB family of inducible transcription factors regulates an array of immune and inflammatory response. Since hsa-miR-939 is downregulated in CRPS and predicted to target inflammatory mediators such as vascular endothelial growth factor A (VEGFA) (upregulated in CRPS), nitric oxide synthase (NOS2A) and transforming growth factor beta1 (TGFB1), the functional role for isolated exosomes is studied using a reporter assay inducible by NFkappaB activation. RAW-Blue cells with chromosomal integration of a secreted embryonic alkaline phosphatase (SEAP) reporter construct that is inducible by NFkappaB and AP-1 (www.invivogen.com/quanti-blue) are used in a colorimetric enzyme assay. Exogenous sense or antisense miR-939 are introduced into exosomes via electroporation. Exosomes are obtained from cell culture media, blood samples of rodent models of inflammatory pain, or CRPS patients. Choice of exosome source is based on 1) ease of obtaining sufficient quantities, 2) profile matching pain state, 3) ease of electroporation and post viability. Functional consequence of miRNA (Wang et al., 2010, RNA Biol. 7:373-380) alteration in exosomes is monitored by adding post-transfected exosomes to the culture media of RAW-Blue cells.

What is claimed is:

1. A method of diagnosing neuropathic pain in a subject, the method comprising:
a. obtaining a biological sample from the subject,
b. determining the level of at least one microRNA or snoRNA in the biological sample,
c. comparing the level of the at least one microRNA or snoRNA in the biological sample with the level of the at least one miRNA or snoRNA in a comparator, wherein when the level of the at least one microRNA or snoRNA in the biological sample is different than the level of the at least one miRNA or snoRNA in the comparator, the subject is diagnosed with neuropathic pain.

2. The method of claim 1, wherein the neuropathic pain is complex regional pain syndrome (CRPS).

3. The method of claim 1, wherein the at least one micro RNA or snoRNA is at least one selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

4. The method of claim 1, wherein the at least one microRNA or snoRNA is at least two microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

5. The method of claim 1, wherein the at least one micro RNA or snoRNA is at least three microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

6. The method of claim 1, wherein the subject is human.

7. The method of claim 1, further comprising the step of treating the subject for neuropathic pain.

8. The method of claim 1, wherein the comparator is at least one comparator selected from the group consisting of positive control, a negative control, a normal control, a wild-type control, a historical control, and a historical norm.

9. The method of claim 1, wherein the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500% by at least 600%, by at least 700%, by at least 800%, by at least 900%, by at least 1000%, by at least 1500%, by at least 2000%, or by at least 5000%.

10. The method of claim 1, wherein the level of the at least one miRNA or snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, or by at least 100%.

11. The method of claim 1, wherein determining the level of the at least one microRNA or snoRNA utilizes at least one technique selected from the group consisting of reverse transcription, PCR and a microarray.

12. A method of determining the severity of neuropathic pain in a subject, the method comprising:
a. obtaining a biological sample from the subject,
b. determining the level of at least one microRNA or snoRNA in the biological sample,
c. comparing the level of the at least one microRNA or snoRNA in the biological sample with the level of the at least one miRNA or snoRNA in a comparator, wherein the greater the difference between the level of the at least one microRNA or snoRNA in the biological sample and the level of the at least one miRNA or snoRNA in the comparator, the greater the severity of neuropathic pain.

13. The method of claim 12, wherein the neuropathic pain is complex regional pain syndrome (CPRS).

14. The method of claim 12, wherein the at least one microRNA or snoRNA is at least one microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

15. The method of claim 12, wherein the at least one microRNA or snoRNA is at least two microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

16. The method of claim 12, wherein the at least one microRNA or snoRNA is at least three microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

17. The method of claim 12, wherein the subject is human.

18. The method of claim 12, further comprising the step of treating the subject for neuropathic pain.

19. The method of claim 12, further comprising the step of stratifying the subject for inclusion in a clinical trial based upon the severity of the subject's neuropathic pain.

20. The method of claim 12, wherein the comparator is at least one comparator selected from the group consisting of positive control, a negative control, a normal control, a wild-type control, a historical control, and a historical norm.

21. The method of claim 12, wherein the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500% by at least 600%, by at least 700%, by at least 800%, by at least 900%, by at least 1000%, by at least 1500%, by at least 2000%, or by at least 5000%.

22. The method of claim 12, wherein the level of the at least one miRNA or snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, or by at least 100%.

23. The method of claim 12, wherein determining the level of the at least one microRNA or snoRNA utilizes at least one technique selected from the group consisting of reverse transcription, PCR and a microarray.

24. A method of evaluating the progression of neuropathic pain in a subject, the method comprising:
a. obtaining a biological sample from the subject,
b. determining the level of at least one microRNA or snoRNA in the biological sample at a first time point,
c. comparing the level of the at least one microRNA or snoRNA in the biological sample at the first time point with the level of the at least one miRNA or snoRNA in a comparator,
d. determining the level of at least one microRNA or snoRNA in the biological sample at a second time point,
e. comparing the level of the at least one microRNA or snoRNA in the biological sample at the second time point with the level of the at least one miRNA or snoRNA in a comparator,
f. wherein when the difference in the level of the at least one microRNA or snoRNA is the biological sample at the second time point, as compared with the comparator, is greater than the difference in the level of the at least one microRNA or snoRNA in the biological sample at the first time point, as compared with the comparator, the neuropathic pain is progressing.

25. The method of claim 24, wherein the neuropathic pain is complex regional pain syndrome (CPRS).

26. The method of claim 24, wherein the at least one microRNA or snoRNA is at least one microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

27. The method of claim 24, wherein the at least one microRNA or snoRNA is at least two microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

28. The method of claim 24, wherein the at least one microRNA or snoRNA is at least three microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

29. The method of claim 24, wherein the subject is human.

30. The method of claim 24, further comprising the step of treating the subject for neuropathic pain.

31. The method of claim 24, further comprising the step of modifying the subject's treatment for neuropathic pain.

32. The method of claim 24, wherein the comparator is at least one comparator selected from the group consisting of a positive control, a negative control, a normal control, a wild-type control, a historical control, and a historical norm.

33. The method of claim 24, wherein the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500% by at least 600%, by at least 700%, by at least 800%, by at least 900%, by at least 1000%, by at least 1500%, by at least 2000%, or by at least 5000%.

34. The method of claim 24, wherein the level of the at least one miRNA or snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, or by at least 100%.

35. The method of claim 24, wherein determining the level of the at least one microRNA or snoRNA utilizes at least one technique selected from the group consisting of reverse transcription, PCR and a microarray.

36. A method of evaluating a treatment of neuropathic pain in a subject in need thereof, the method comprising:
a. obtaining a biological sample from the subject,
b. determining the level of at least one microRNA or snoRNA in the biological sample at a first time point,
c. comparing the level of the at least one microRNA or snoRNA in the biological sample at the first time point with the level of the at least one miRNA or snoRNA in a comparator,
d. determining the level of at least one microRNA or snoRNA in the biological sample at a second time point,
e. comparing the level of the at least one microRNA or snoRNA in the biological sample at the second time point with the level of the at least one miRNA or snoRNA in a comparator,
f. wherein when the difference in the level of the at least one microRNA or snoRNA is the biological sample at the second time point, as compared with the comparator, is greater than the difference in the level of the at least one microRNA or snoRNA in the biological sample at the first time point, as compared with the comparator, the treatment of neuropathic pain is reducing neuropathic pain.

37. The method of claim 36, wherein the neuropathic pain is complex regional pain syndrome (CPRS).

38. The method of claim 36, wherein the at least one micro RNA, or snoRNA is at least one microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

39. The method of claim 36, wherein the at least one microRNA or snoRNA is at least two microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

40. The method of claim 36, wherein the at least one microRNA or snoRNA is at least three microRNA selected from the group consisting of hsa-miR-939, hsa-miR-25#, hsa-let-7c, hsa-let-7a, hsa-let-7b, hsa-miR-320B, hsa-miR-126, hsa-miR-629.A, hsa-miR-664, hsa-miR-320, hsa-miR-1285, hsa-miR-625#, hsa-miR-532-3p, hsa-miR-181a-2#, RNU48, hsa-miR-720, RNU44, and hsa-miR-1201.

41. The method of claim 36, wherein the subject is human.

42. The method of claim 36, further comprising the step of continuing to treat the subject for neuropathic pain.

43. The method of claim 36, further comprising the step of modifying the subject's treatment for neuropathic pain.

44. The method of claim 36, wherein the comparator is at least one comparator selected from the group consisting of a positive control, a negative control, a normal control, a wild-type control, a historical control, and a historical norm.

45. The method of claim 36, wherein the level of the at least one miRNA or snoRNA is higher than the level of the at least one miRNA or snoRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500% by at least 600%, by at least 700%, by at least 800%, by at least 900%, by at least 1000%, by at least 1500%, by at least 2000%, or by at least 5000%.

46. The method of claim 36, wherein the level of the at least one miRNA or snoRNA is lower than the level of the at least one miRNA or snoRNA in the comparator by at least 10%, by at least 20%, by at least 30%, by at least 40%, by at least 50%, by at least 60%, by at least 70%, by at least 80%, by at least 90%, or by least 100%.

47. The method of claim 36, wherein determining the level of the at least one microRNA or snoRNA utilizes at least one technique selected from the group consisting of reverse transcription, PCT and a microarray.
